(19) United States
(12) Patent Application Publication
(10) Pub. No.: US 2024/0074954 A1
(43) Pub. Date: Mar. 7, 2024

(54) CALCIUM HYDROXYAPATITE PARTICLES AND USE THEREOF

(71) Applicant: MERZ NORTH AMERICA, Raleigh, NC (US)

(72) Inventors: Bartosch NOWAG, Wiesbaden (DE); Roland STRAGIES, Berlin (DE); Thomas HENGL, Frankfurt Am Main (DE); Dean ERICKSON, Raleigh, NC (US); Tim LIGMAN, Raleigh, NC (US); Christopher GROPPI, Raleigh, NC (US)

(21) Appl. No.: 18/262,945

(22) PCT Filed: Jan. 25, 2022
(86) PCT No.: PCT/US2022/013630

§ 371 Date: Jul. 26, 2023

(30) Foreign Application Priority Data
Jan. 26, 2021 (EP) 21153473.0

(51) Int. Cl. A61K 8/24; A61K 8/02; A61K 9/00; A61K 33/42; A61K 45/06; A61Q 19/08; C01B 25/32

(52) U.S. Cl. CPC A61K 8/24; A61K 8/025; A61K 9/0019; A61K 33/42; A61K 45/06; A61Q 19/08; C01B 25/32; A61K 2800/91; C01P 2004/32; C01P 2004/51; C01P 2004/61

(57) ABSTRACT

The invention relates to calcium hydroxyapatite particles having been sintered at a certain temperature range and which are not treated at a temperature above this range. Furthermore, the present invention relates to an injectable composition comprising such particles and to uses thereof. Surprisingly, it was found that the particles of the invention are superior over calcium hydroxyapatite particles known in the art with respect to bio-stimulation.

The present invention relates to calcium hydroxyapatite particles having been sintered at a certain temperature range, which are not treated at a temperature above this range. Preferably, the present invention relates to calcium hydroxyapatite particles having been sintered at a temperature in the range of from 910° C. to 1030° C. Preferably, the particles are not treated at a temperature of more than 1030° C. Furthermore, the present invention relates to an injectable composition comprising such particles and to uses thereof. Surprisingly, it was found that the particles of the invention are superior over calcium hydroxyapatite particles known in the art with respect to biostimulation.

In recent years, facial and body re-shaping, in particular reduction of undesired wrinkles, has gained increasing interest. For example, filling of wrinkles, breast reconstruction or augmentation, rejuvenation of the skin, soft tissue augmentation of other kind, are frequently performed. In order to avoid the need of surgical interventions in this context, a number of different dermal fillers can be injected subcutaneously or within the deeper layers of the skin.

For several of these purposes, filler materials have been used. A major drawback of many filler components is, however, that either biodegradation of such materials is rapid and the filler material is not suitable for long term solutions or that there is no biodegradation but rather a defensive reaction of the administered subject's body. Many filler materials are xenobiotics, which are not tissue-like.

It is desirable that at least a part of the filled tissue area is finally filled by the administered subject's own tissue and/or extracellular matrix. This can be achieved by products, such as injectable calcium hydroxyapatite (CaHA, Ca₅(PO₄)₃(OH)) particles. Such fillers are e.g. described in U.S. Pat. No. 6,537,574 and WO 2001/012247. A commercial product comprising calcium hydroxyapatite particles is Radiesse® (Merz Pharmaceuticals GmbH, Frankfurt, Germany). Radiesse® is a dermal filler with excellent biostimulation properties. The filler comprises calcium hydroxyapatite particles and a sodium carboxy methyl cellulose (NaCMC) gel carrier. The injection of Radiesse® can lead to neocollagenesis by stimulation of fibroblasts.

The potential of those calcium hydroxyapatite particles to stimulate fibroblasts is essential on the amount of collagen produced. In the administered subject, collagen production is stimulated. This can lead to a long-term filling effect of wrinkles replacing the degraded calcium hydroxyapatite particles by collagen. WO 2008/088381 teaches the use of thickened compositions comprising calcium hydroxyapatite particles, which have been sintered at 1150° C. U.S. Pat. No. 6,537,574 and WO 2001/012247 describe that injectable calcium hydroxyapatite particles should be prepared by shaping the particles and subjecting these to a sintering step at about 1050 to 1200° C. for at least one hour. Optionally, also a presintering step at about 800 to 1000° C. for about one hour can be additionally applied to minimize agglomeration. Treatment of calcium hydroxyapatite nanocrystal needles of less than 100 nm in length at 900° C. is described in Eslami et al., (Iranian Journal of Pharmaceutical Sciences, 2008, 4(2):127-134).

Nieh et al. (“Synthesis and characterization of porous hydroxyapatite and hydroxyapatite coatings”, Conference: 2001 Minerals, Metals & Materials Society Annual Meeting & Exhibition, New Orleans, LA (US), Feb. 11-15, 2001) teaches preparing calcium hydroxyapatite coatings and large-sized particles sintered at different temperatures which preferably have pore sizes of 100 to 200 μm which are usable as bone models and coatings for implants.

Several positive effects of Radiesse® are summarized in van Loghem et al. (The Journal of Clinical Aesthetic Dermatology, 2015, 8(1):38-49). Wrinkles can be effectively treated by administering subjects with Radiesse® (calcium hydroxyapatite particles) subdermally. It was shown that facial augmentation is possible with such product, (see Jacovella, Clinical Interventions in Aging, 2008, 3:161-174). Collagen is generated by fibroblasts in the tissue area, administered with the calcium hydroxyapatite particles without significant undesired side effects (cf. Coleman et al., Dermatologic Surgery, 2008, 34:S53-S55; Berlin et al., Dermatologic Surgery, 2008, 34:S64-S67).

The administration of calcium hydroxyapatite particles was found to have a long-lasting, volume increasing effect, even after calcium hydroxyapatite is degraded. Collagen production is stimulated. It was found that, several months after injection, collagen types I and III were significantly increased (Yutskovskaya and Kogan, Journal of Drugs in Dermatology, 2017, 16:68-74).

Dermal fillers, which are pharmaceutically/cosmetically acceptable, lead to fillings with the subject's own tissue and/or extracellular matrix and lead to a long-term activity are of interest. It is particularly desired to improve calcium hydroxyapatite particles further. It is e. g. desired to further enhance bioactivity such as the collagen stimulating effect of dermal fillers.

Surprisingly, it has been found that calcium hydroxyapatite particles which have been sintered, but not subjected to a temperature of more than 1030° C., bear a particularly high collagen stimulating effect.

Accordingly, a first aspect of the present invention relates to calcium hydroxyapatite particles which have been sintered at a temperature in the range of from 910 to 1030° C. and have not been subjected to a temperature of more than 1030° C.

Accordingly, there may be no heating to a temperature of more than 1030° C., which may further alter the material properties, preferably not heated above the indicated sintering temperature. The present invention also relates to calcium hydroxyapatite particles which have been sintered at a temperature in the range of from 910 to 1030° C. and have not been subjected to a temperature of more than 1030° C., wherein the weight average particle diameter is from 1 to 500 μm as determined by sieving or light scattering.

The calcium hydroxyapatite particles of the present invention may have a well-defined higher surface area which may enable increase of the collagen synthesis of fibroblasts and, thus, bears particularly high biostimulation.

As used herein, the terms “calcium hydroxyapatite”, “calcium hydroxylapatite” and “basic calcium orthophosphate”, “calcium hydroxyphosphate”, “calcium phosphate tribasic”, “hydroxyapatite”, “hydroxylapatite”, and “tribasic calcium phosphate” and the abbreviations “CaHA” and “HAp” should be interchangeably understood in the broadest sense as commonly understood in the art. Calcium hydroxyapatite may be expressed by the formulae Ca₅(PO₄)₃(OH) and Ca₅[OH|(PO₄)₃], respectively.

The calcium hydroxyapatite particles may have any shape. These may be spherical, ellipsoid, crystalline, random (also: irregular), or a mixture of two or more thereof. In a preferred embodiment, the calcium hydroxyapatite particles are (essentially) spherical or (essentially) ellipsoid. In a preferred embodiment, the calcium hydroxyapatite particles are (essentially) spherical.

In a preferred embodiment, the calcium hydroxyapatite particles are spherical having a D-ratio above 0.7. Thus, the calcium hydroxyapatite particles preferably have a D-ratio above 0.7, more preferably above 0.8, in particular above 0.9. In this context, a D-ratio of 1.0 indicates perfect roundness.

As used herein, the term “spherical” may be understood in the broadest sense as being substantially globular or ball-shaped, respectively. This does not necessarily mean perfect spheres, but characterizes the particles as not having sharp or angular edges. Preferably, the D-ratio above 0.7 more preferably above 0.8, in particular above 0.9. Thus, the extensions in all three spatial directions is typically substantially the same.

The D-ratio may be determined by any means. As used herein, it is typically determined by means of microscopic imaging (also: by microscopy). For this purpose, microscopic images of individual particles are taken. Software conducts the measurements.

In a preferred embodiment, the calcium hydroxyapatite particles of the calcium hydroxyapatite particles have porous surfaces. Accordingly, the surfaces of the calcium hydroxyapatite particles are preferably not smooth and not having a tiled appearance. Preferably, the surfaces of the calcium hydroxyapatite particles bear numerous cavities. Preferably, also the inner of the calcium hydroxyapatite particles bear numerous pores/cavities. Thus, the calcium hydroxyapatite particles are preferably porous.

The pores may be of any dimension. In a preferred embodiment, the surfaces of the calcium hydroxyapatite particles have pores of an average diameter between 10 and 500 nm at the surface as determined by Hg-porosimetry. Alternatively, the average diameter may be determined by microscopy. In a preferred embodiment, the surfaces of the calcium hydroxyapatite particles have pores of a diameter between 10 and 100 nm diameter at the surface as determined by Hg-porosimetry. In a preferred embodiment, the surfaces of the calcium hydroxyapatite particles have pores of an average diameter between 10 and 400 nm at the surface as determined by Hg-porosimetry. Preferably, the surfaces of the calcium hydroxyapatite particles have pores of an average diameter between 20 and 300 nm, or between 30 and 250 nm, or between 50 and 220 nm, at the surface as determined by Hg-porosimetry.

Preferably, each calcium hydroxyapatite particle bears at least 10, or at least 100 of such pores at its surface. It will be understood that the presence of such pores does not exclude the optional presence of one or more pores having other dimensions. Microscopy usable for the determination of pores is preferably scanning electron microscopy (SEM).

The calcium hydroxyapatite particles may have any particle size. Preferably, in the context of the present invention, the calcium hydroxyapatite particles are suitable for being injected. In other words, the calcium hydroxyapatite particles preferably are injectable calcium hydroxyapatite particles. Therefore, the calcium hydroxyapatite particles typically have a mean particle diameter in the micrometer range (also: are microspheres) and, thus, have a mean particle diameter in the range of 1 to 1000 μm.

In a preferred embodiment, the calcium hydroxyapatite particles have a mean particle diameter of from 1 to 500 μm, or of from 5 to 500 μm, 1 to 150 μm, or of from 2 to 100 μm, or of from 5 to 80 μm, or of from 10 to 60 μm, or of from 15 to 50 μm, or of from 20 to 45 μm, or of from 25 to 45 μm, as determined by light scattering.

In a preferred embodiment, the calcium hydroxyapatite particles have a weight average particle diameter of from 1 to 500 μm, or of from 1 to 500 μm 1 to 150 μm, or of from 2 to 100 μm, or of from 5 to 80 μm, or of from 10 to 60 μm, or of from 15 to 50 μm, or of from 20 to 45 μm, or of from 25 to 45 μm, as determined by sieving or light scattering.

Preferably, at least 80% by weight of the total mass of calcium hydroxyapatite particles is represented by calcium hydroxyapatite particles falling in an above size range and/or at least 80% of calcium hydroxyapatite particles in number fall within the above size ranges.

As used herein, particle size may be determined by any means (e.g., light scattering (light diffraction), sieving, microscopy, etc.). The number values shown herein refer to the (volume) mean size range determined by light scattering (PSA).

In a preferred embodiment, particle size is determined by light scattering. In an alternative embodiment, particle size is determined by sieving, i.e., by test sieves and mechanically sieving the sample and weigh the fractions to determine what weight % is above or below the test sieve used.

The calcium hydroxyapatite particles may have been sintered at any temperature in the range or from 910 to 1030° C., wherein the calcium hydroxyapatite particles have preferably not been subjected to temperatures above the sintering temperature.

In a preferred embodiment, the calcium hydroxyapatite particles have been sintered at a temperature in the range of from 910 to 995° C., or of from 920 to 995° C., or from 930 to 990° C., or from 940 to 985° C., or from 950 to 980° C., or from 960 to 975° C., and wherein the calcium hydroxyapatite particles have not been subjected to temperatures above the temperatures of the sintering temperature.

In a preferred embodiment, the particles are not subjected to a temperature of more than 995° C., or of more than 985° C., or of more than 980° C., or of more than 975° C. In a preferred embodiment, the particles are not subjected to a temperature of more than the maximal temperature of the sintering.

The sintering time to which the calcium hydroxyapatite particles have been subjected may be adapted to the sintering temperature and/or the mean particle diameter. Preferably, the calcium hydroxyapatite particles have been sintered for several hours. Preferably, the calcium hydroxyapatite particles have been sintered until uniform solid particles are obtained. In a preferred embodiment, the calcium hydroxyapatite particles have been sintered for 1 to 24 hours. In a preferred embodiment, the calcium hydroxyapatite particles have been sintered for 2 to 12 hours, or 3 to 16 h In a preferred embodiment, the calcium hydroxyapatite particles have been sintered for 1 hour to 2 hours, 1 hour to 3 hours, 2 to 4 hours, 3 to 5 hours, 4 to 6 hours, 5 to 7 hours, 6 to 8 hours, 7 to 9 hours, 8 to 10 hours, 9 to 11 hours, 10 to 12 hours, 11 to 13 hours, 12 to 14 hours, 13 to 15 hours, 14 to 16 hours, or 12 to 24 hours.

In a preferred embodiment, the calcium hydroxyapatite particles have been sintered for 1 to 24 hours at a temperature of from 960 to 975° C. In a preferred embodiment, the calcium hydroxyapatite particles having a mean particle diameter in the range of from 25 to 45 μm, as determined by light scattering, have been sintered for 1 to 24 hours at a temperature of from 960 to 975° C.

In a preferred embodiment, the calcium hydroxyapatite particles having a mean particle diameter in the range of from 25 to 45 μm, as determined by light scattering, and having pores of a diameter of 10 to 500 nm on their surface, as determined by microscopy, have been sintered for 1 to 24 hours at a temperature of from 960 to 975° C.

Optionally, the calcium hydroxyapatite particles may comprise one or more other metal ions besides calcium in the CaHA particle crystal structure, such as a metal ion selected from the group consisting of fluorine, sodium, lithium, potassium, silicon, magnesium, and a combination of two or more thereof. This may optionally result in a positive effect on neocollagenesis.

The calcium hydroxyapatite particles may be prepared by any means known in the art. Suitable procedures are, for example, described in U.S. Pat. No. 6,537,574 and WO 2001/012247. In a preferred embodiment, a slurry of small-sized calcium hydroxyapatite grains/crystals may be spray-dried.

Such slurry may have any content of calcium hydroxyapatite usable for the purpose of preparing calcium hydroxyapatite by spray-drying. In one embodiment, the content of calcium hydroxyapatite in the slurry is set to 5 to 80% by weight, to 10 to 60% by weight, or to 20 to 40% by weight.

In a preferred embodiment, for this purpose, the slurry may be pumped through a nozzle to form spherical particles that may be led through a column of heated air to remove the moisture. The size of the particles may be set by the choice of the nozzle. He particle size may be further improved by sieving different fractions.

The obtained un-sintered particles may be sintered at the desired temperature, according to the invention a temperature in the range of from 910 to 1030° C. as defined herein, for several hours, until the sintering has baked the previous submicron grains/crystals into uniform solid particles. Thus, the grains/crystals typically fuse and, thereby, enhance hardness. In a preferred embodiment, the sintering time is in the range of from 1 hour to 24 hours.

In a preferred embodiment, the sintering time is in the range of from 1 hour to 2 hours, 1 hour to 3 hours, 2 to 4 hours, 3 to 5 hours, 4 to 6 hours, 5 to 7 hours, 6 to 8 hours, 7 to 9 hours, 8 to 10 hours, 9 to 11 hours, 10 to 12 hours, 11 to 13 hours, 12 to 14 hours, 13 to 15 hours, 14 to 16 hours, or 12 to 24 hours. The size of the particles may be further improved by sieving different fractions.

As used herein, the slurry of submicron grains/crystals of calcium hydroxyapatite particles usable for preparing the calcium hydroxyapatite particles may be prepared by any means. For instance, it may be prepared by elutriating optionally commercially available calcium hydroxyapatite powder of a submicron grain size in water or an aqueous buffer or an aqueous and/or organic solution. Alternatively or additionally, the grains/crystal or preferably submicron size may also be prepared.

This may be achieved by admixing one or more soluble solutions of soluble calcium salt (e.g., calcium nitrate, calcium chloride, etc.) and a one or more soluble solutions of soluble hydrogen phosphate or dihydrogen phosphate (e.g., diammonium hydrogen phosphate). The mixing may be performed under vigorous mixing in order to obtain small-sized grains/crystals. Optionally, the pH may be adjusted to basic pH. Then, a slurry may be directly obtained. Optionally, the slurry may also be aged for several hours. Optionally, the crystal may be washed by one or more centrifugation/washing steps. A detailed procedure for obtaining small-sized calcium hydroxyapatite crystals is described in Eslami et al., (Iranian Journal of Pharmaceutical Sciences, 2008, 4(2):127-134). Optionally, the slurry may further comprise of one or more wetting agents and/or binders such as polysorbate, sodium oxalate, polyvinyl alcohol, dextrin and/or carbonwax may be added.

Optionally, the calcium hydroxyapatite particles may comprise in the inside and/or may be coated with one or more agents stimulating neocollagenesis such as, e.g., polypeptides and/or small-molecular weight compounds stimulating neocollagenesis.

As indicated above, the calcium hydroxyapatite particles of the present invention may be administerable to a subject by means of injection, in particular to a subject's skin and/or soft tissue.

Accordingly, a further aspect of the present invention relates to an injectable composition comprising (or consisting of):
(A) one or more types of calcium hydroxyapatite particles of the present invention as component A; and
(B) one or more pharmaceutically acceptable carriers as component B.
(C) optionally one or more local anesthetics as component C; and
(D) optionally one or more pharmaceutically acceptable additives other than components A, B and C, as component D.

It will be understood that the definitions and preferred embodiments as laid out in the context of the calcium hydroxyapatite particles of the present invention mutatis mutandis apply to the injectable composition.

As used herein, the terms “component” and “ingredient” may be understood interchangeably in the broadest sense as a part of the composition of the present invention.

As used herein, the term “pharmaceutically acceptable” may be understood in the broadest sense as any being reasonably usable in a pharmaceutical and/or cosmetic context. It will be understood that a pharmaceutically acceptable component or composition will typically also be inherently usable as being cosmetically acceptable. A pharmaceutically acceptable component or composition bears a low toxicity and can be administered to a human or animal (typically mammal) body without seriously harm this human or animal.

Preferably, the injectable composition of the present invention is a pharmaceutically and/or cosmetically acceptable composition. The composition of the present invention may have any galenic form. In one embodiment of the present invention, the injectable composition of the present invention is liquid or viscous. In another to embodiment, the injectable composition of the present invention is pasty. Such composition can be considered as a dermal filler.

In a preferred embodiment, the injectable composition is injectable into the skin or into other soft tissue. Preferably, the injectable composition is usable for skin or other soft tissue improvement. In a preferred embodiment, injectable composition is injectable (sub)cutaneously/(sub)dermally. Preferably, the injectable composition is suitable for injection into a mammal, in particular a human. Preferably, the composition of the present invention is preferably (essentially) sterile and is preferably a-pyrogenic.

As used herein, the terms “liquid”, “viscous” and “pasty” may be understood in accordance with general understanding in the art. Preferably, “liquid” as used in the context of the present invention means having a viscosity of less than 10 mPas (millipascal-seconds, at standard conditions, 20° C., at 1013.25 hPa).

Preferably, “viscous” as used in the context of the invention means having a viscosity of from 10 to 1000 mPas (at standard conditions, 20° C., at 1013.25 hPa). The terms “viscous”, “gel” and “gel-like” should be understood interchangeably. Preferably, “pasty” as used in the context of the present invention means having a viscosity of from 1000 to 1,000,000 mPas (at standard conditions, 20° C., at 1013.25 hPa). These viscosity values can be determined by any means, for example, by a rotational/oscillating viscometer, e.g., according DIN 53019-4:2016-10). According to the invention, when the injectable composition is a liquid, viscous or pasty injectable composition, the calcium hydroxyapatite particles (component A) are preferably dispersed in the injectable composition, i.e., in the liquid, viscous or pasty component of the injectable composition. Accordingly, a liquid, viscous or pasty injectable composition is typically a dispersion.

The injectable composition of the present invention comprises one or more pharmaceutically acceptable carriers as component B, wherein at least one carrier preferably is a pasty, viscous or liquid carrier.

A pharmaceutically acceptable carrier (component B) according the present invention may be any carrier that is pharmaceutically acceptable, therefore, any carrier that is (essentially) non-toxic to the human or animal (typically mammal) body.

The one or more pasty, viscous or liquid carriers may be any pharmaceutically acceptable carrier that is pasty, viscous or liquid. For instance, the one or more pasty, viscous or liquid carriers may optionally comprise one or more pharmaceutically acceptable solvents such as, e.g., glycerol, water, an aqueous buffer (e.g., a saline or phosphate buffered saline), dimethyl sulfoxide (DMSO), ethanol, vegetable oil, paraffin oil, or combinations thereof. In one embodiment of the present invention, the one or more pasty, viscous or liquid carriers may comprise or consist of an a-pyrogenic isotonic buffer, such as a physiological saline solution or a buffered physiological saline solution.

In a preferred embodiment, the sum of all pharmaceutically acceptable carriers (component B) comprises at least 10% by weight, at least 20% by weight, at least 50% by weight, at least 60% by weight, at least 70% by weight, or at least 80% by weight, or at least 90% by weight, referred to component B, of one or more pasty, viscous or liquid carriers.

As used herein, a content by weight (e.g.,% by weight) typically refers to the component as such. In case of a solid matter, it typically refers to the dry matter of the respective component (% by weight, referred to dry matter).

In one embodiment of the present invention, the pharmaceutically acceptable carrier (component B) comprises at least 50% by weight, at least 60% by weight, at least 70% by weight, or at least 80% by weight, or at least 90% by weight, referred to component B, of glycerol. The one or more types of calcium hydroxyapatite particles (component A) may be dispersed in this carrier.

As used herein, the percentages by weight (% by weight) and the weight ratios of components are typically referred to the dry matters of the components. The terms “dry matter”, “dry weight” and “solid contend” may be understood interchangeably in the broadest sense as generally understood in the art. The person skilled in the art will notice that the dry matter may refer to the weight of the respective component without solvents/diluents and other components. The dry matters may also be considered when the respective components are dissolved, suspended or are forming a (hydro)gel with other components. In this case, the theoretical dry matter may be calculated, i.e., the weight of the solvents/diluents and further components may be substracted from the total weight.

In a preferred embodiment, the one or more pharmaceutically acceptable carriers (component B) are selected from the group consisting of one or more polysaccharide derivatives or pharmaceutically acceptable salts thereof, one or more polysaccharides or pharmaceutically acceptable salts thereof, glycerol, water, one or more aqueous buffers, and combinations or two or more thereof.

In a preferred embodiment, the one or more pharmaceutically acceptable carriers (component B) comprise one or more liquid, viscous or pasty components such as, e.g., glycerol, water, one or more aqueous buffers, and combinations or two or more thereof. This may make the injectable composition injectable.

The one or more polysaccharide or derivatives thereof or pharmaceutically acceptable salts may preferably have thickening properties. The one or more polysaccharide or derivatives thereof or pharmaceutically acceptable salts thereof may have any molecular weight. Preferably, their molecular weight is in the range of from 1 kDa to 10 MDa, more preferably from 5 kDa to 5 MDa. Also a mixture of polysaccharides or derivatives or salts thereof may be used. Such mixture may be of the same or different type of polysaccharides or derivatives or salts thereof and may have different molecular size. Polysaccharides or derivatives thereof or pharmaceutically acceptable salts thereof may be non-crosslinked or cross-linked.

As used herein, mean molecular weight may be determined by any routine means suitable for this purpose such as, e.g., gel permeation chromatography (GPC), size exclusion chromatography (SEC), measuring the thickening effect (viscosimetry), mass spectrometry, etc. The mean molecular masses of the soluble fraction of polysaccharides or derivatives thereof or pharmaceutically acceptable salts thereof are preferably determined by gel permeation chromatography (GPC). The mean molecular masses of the insoluble, gel-forming fraction of polysaccharides or derivatives thereof or pharmaceutically acceptable salts thereof are preferably determined by measuring the thickening effect (viscosimetry) by routine experiments (e.g., at 25° C. by an EP monograph method on an Ubbelohe viscometer). As used herein, 1000 kDa (kilodaltons) are 1 MDa (megadalton).

The polysaccharide or derivative or pharmaceutically acceptable salt thereof may optionally form a gel in combination with the one or more pasty, viscous or liquid pharmaceutically acceptable carriers. The polysaccharide or derivative or pharmaceutically acceptable salt thereof may optionally form a hydrogel in combination with the one or more pasty, viscous or liquid carriers. The polysaccharide or derivative or pharmaceutically acceptable salt thereof may optionally be partly or completely dissolved the one or more viscous or liquid carriers. The one or more types of calcium hydroxyapatite particles (component A) to may be dispersed in the one or more pasty, viscous or liquid carriers.

In a preferred embodiment, component B comprises or consists of:
(B1) one or more liquid, viscous or pasty pharmaceutically acceptable carriers, in particular liquid, viscous or pasty pharmaceutically acceptable carriers selected from the group consisting of glycerol, water, one or more aqueous buffers, and combinations or two or more thereof; and
(B2) one or more solid pharmaceutically acceptable carriers, preferably one or more polysaccharides or derivatives thereof or pharmaceutically acceptable salts thereof, in particular polysaccharides or derivatives selected from the group consisting of cellulose derivative (e.g., carboxymethyl cellulose, carboxyethyl cellulose), cellulose, and mixtures of two or more thereof.

Component B may comprise components B1 and B2 in any content ratio. In a preferred embodiment, component B comprises or consists of:
0.1 to 99% by weight, or 50 to 99.9% by weight, or 75 to 99% by weight, referred to component B, of B1, and
0.1 to 99% by weight, or 0.1 to 50% by weight, or 1 to 25% by weight, referred to component B, of B2.

In a preferred embodiment, the one or more pharmaceutically acceptable carriers (component B) are selected from the group consisting of (one or more types of) carboxymethyl cellulose or pharmaceutically acceptable salts thereof, glycerol, water, one or more aqueous buffers, and combinations or two or more thereof.

In a preferred embodiment, the one or more pharmaceutically acceptable carriers (component B) comprise (or consists of) (one or more types of) carboxymethyl cellulose or pharmaceutically acceptable salts thereof and glycerol.

As noted above, the composition of the present invention may optionally comprise one or more local anesthetics as component C. Thus, in one embodiment of the present invention, the injectable composition comprises one or more local anesthetics (component C).

A local anesthetic (component C) may be any local anesthetic. Preferably, a local anesthetic (component C), if present, is selected from the group consisting of: lidocaine, ambucaine, amolanone, amylocaine, benoxinate, benzocaine, betoxycaine, biphenamine, bupivacaine, butacaine, butamben, butanilicaine, to butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and combinations of two or more thereof and salts thereof.

Alternative local anesthetics and combinations and salts thereof may also be used as component C. In a preferred embodiment, the local anesthetic (component C) is or comprises lidocaine. Combinations of two or more of the mentioned anesthetic agents, for example a combination of lidocaine and other “caine”-anesthetics like prilocaine, may also be used herein. A local anesthetic may make injection into a subject more comfortable.

As noted above, the composition of the present invention may optionally comprise one or more pharmaceutically acceptable additives other than components A, B and C as component D. Thus, in one embodiment of the present invention, the injectable composition comprises one or more pharmaceutically acceptable additives other than components A, B and C (component D).

Such pharmaceutically acceptable additive (component D) may be any further agent that is (essentially) non-toxic to the human or animal (typically mammal) body. Such pharmaceutically acceptable additive (component D) may optionally be a bioactive ingredient that has an impact on biostimulation such as on collagen production (neocollagenesis factor) and/or cell proliferation (cell proliferation factor). Such pharmaceutically acceptable additive (component D) may also be an ingredient not having biostimulating activity.

For instance, pharmaceutically acceptable additive (component D) may be a bioactive ingredient selected from the group consisting of one or more agents stimulating neocollagenesis such as, e.g., one or more hyaluronic acids or pharmaceutically acceptable salts thereof, one or more polypeptides or pharmaceutically acceptable salts thereof, and one or more small-molecular weight compounds stimulating neocollagenes or pharmaceutically acceptable salts thereof.

The one or more hyaluronic acids or pharmaceutically acceptable salts thereof, one or more polypeptides or pharmaceutically acceptable salts thereof may have any molecular weight.

Preferably, the one or more hyaluronic acids or pharmaceutically acceptable salts thereof may have a molecular weight is in the range of from 1 kDa to 10 MDa, more preferably in the range of from 5 kDa to 5 MDa, or in the range of from 0.3 MDa to MDa, or in the range of from 0.3 MDa to 1 MDa, or in the range of from 1 MDa to MDa. It will be understood that also a mixture of hyaluronic acids or pharmaceutically acceptable salts thereof may be used. Such mixture may have different molecular size. Hyaluronic acids or pharmaceutically acceptable salts thereof may be non-crosslinked or cross-linked or may be a mixture of crosslinked and non-crosslinked.

Preferably, the one or more polypeptides or pharmaceutically acceptable salts thereof may have a molecular weight in the range of from 0.5 kDa to 500 kDa.

A small-molecular weight compound preferably has a molecular weight of not more than 1000 Da, of not more than 750 Da, or of not more than 500 Da. A cell proliferation factor may improve cellular invasion into the administered composition of the present invention.

For instance, pharmaceutically acceptable additive (component D) may be an ingredient not having biostimulating activity. Such pharmaceutically acceptable additive (component D) may exemplarily be selected from the group consisting of one or more detergents (e.g., sodium lauryl sulfate (SLS)/sodium doceyl sulfate (SDS)), one or more coloring agents (e.g., TiO₂, food coloring), one or more vitamins, one or more salts (e.g., sodium, potassium, magnesium, calcium, and/or zinc salts), one or more humectants (e.g., sorbitol, glycerol, mannitol, propylene glycol, polydextrose), one or more enzymes, one or more preserving agents (e.g., benzoic acid, methylparabene), one or more texturing agents (e.g., polyethylene glycol (PEG), sorbitol), one or more emulsifiers, one or more separating agents, one or more antioxidants, one or more herbal and plant extracts, one or more stabilizing agents, one or more polymers (e.g., hydroxypropyl methacrylamide (HPMA), polyethylene imine (PEI), polyethylene glycol (PEG)), one or more uptake mediators (e.g., polyethylene imine (PEI), dimethyl sulfoxide (DMSO), a cell-penetrating peptide (CPP), a protein transduction domain (PTD), an antimicrobial peptide, etc.) one or more antibody/antibodies, one or more counterstain dyes (e.g., fluorescein, to fluorescein derivatives, Cy dyes, an Alexa Fluor dyes, S dyes, rhodamine, quantum dots, etc.), one or more cell proliferation factors, one or more homeopathic ingredients, and combinations of two or more thereof.

A dye may either improve localization of the injection (e.g., a pharmaceutically acceptable fluorescent dye like fluorescein or rhodamine) or may improve invisibility of the otherwise whitish composition of the present invention (e.g., by rendering it flesh-colored).

The optional further components C and/or D may be partly or completely comprised in the liquid, viscous or pasty component of the injectable composition or may be dispersed therein. In a preferred embodiment, the injectable composition of the present invention is a gel. Thus, it is preferably a gel-like, i.e., pasty or viscous, injectable composition.

The components A and B and optionally C and optionally D may be comprised in the injectable composition in any content ranges and ratios.

In a preferred embodiment, the injectable composition comprises at least 1% by weight, or at least 5% by weight, or at least 10% by weight, or at least 20% by weight, or at least 30% by weight, or at least 40% by weight, or at least 50% by weight, referred to the injectable composition, of one or more types of calcium hydroxyapatite particles as component A.

In a preferred embodiment, the injectable composition comprises 1 to 80% by weight, 5 to 90% by weight, 10 to 80% by weight, 20 to 77% by weight, 30 to 75% by weight, 40 to 73% by weight, 50 to 72% by weight, 50 to 80% by weight, or 55 to 70% by weight, referred to the injectable composition, of one or more types of calcium hydroxyapatite particles as component A. The weight percentages related to component A refer to dry matter of component A.

In a preferred embodiment, the injectable composition comprises up to 80% by weight, 1 to 80% by weight, 2 to 75% by weight, 3 to 70% by weight, 4 to 65% by weight, 5 to 60% by weight, 10 to 55% by weight, 20 to 50% by weight, or 30 to 50% by weight, referred to the injectable composition, of one or more types of pharmaceutically acceptable carriers as component B.

In a preferred embodiment, the injectable composition comprises up to 10% by weight, 0.001 to 10% by weight, 0.001 to 5% by weight, 0.01 to 3% by weight, or 0.1 to 2% by weight, referred to the injectable composition, of one or more local anesthetics as component C. In case component C in pure form is a solid compound, the weight percentages related to component D may refer to dry matter of component C.

In a preferred embodiment, the injectable composition comprises up to 10% by weight, 0.001 to 10% by weight, 0.01 to 5% by weight, or 0.1 to 2% by weight, referred to the injectable composition, of one or more pharmaceutically acceptable additives other than components A, B and C as component D. In case component D in pure form is a solid compound, the weight percentages related to component D may refer to dry matter of component D.

In a preferred embodiment, the injectable composition comprises (or consists of):
(A) 1 to 80% by weight, referred to dry matter, referred to the injectable composition, of one or more types of calcium hydroxyapatite particles as component A;
(B) 1 to 80% by weight, referred to the injectable composition, of one or more pharmaceutically acceptable carriers as component B;
(C) 0 to 10% by weight, referred to the injectable composition, of one or more local anesthetics as component C; and
(D) 0 to 50% by weight, referred to the injectable composition, of one or more pharmaceutically acceptable additives other than components A, B, C and D as component D.

In a preferred embodiment, the injectable composition comprises (or consists of):
(A) 1 to 80% by weight, referred to dry matter, referred to the injectable composition, of one or more types of the calcium hydroxyapatite particles of the present invention as component A;
(B) 1 to 80% by weight, referred to the injectable composition, of one or more pharmaceutically acceptable carriers as component B comprising at least one pasty, viscous or liquid carrier;
(C) 0 to 10% by weight, referred to the injectable composition, of one or more local anesthetics as component C; and
(D) 0 to 50% by weight, referred to the injectable composition, of one or more pharmaceutically acceptable additives other than components A, B, C and D as component D.

In a preferred embodiment, the injectable composition comprises (or consists of):
(A) 1 to 80% by weight, referred to dry matter, referred to the injectable composition, of one or more types of calcium hydroxyapatite particles having a mean particle diameter of from 15 to 50 μm as component A;
(B) 1 to 80% by weight, referred to the injectable composition, of one or more pharmaceutically acceptable carriers as component B comprising or consisting of:
   (B1) 50 to 99.9% by weight of one or more liquid, viscous or pasty pharmaceutically acceptable carriers, in particular liquid, viscous or pasty pharmaceutically acceptable carriers selected from the group consisting of glycerol, water, one or more aqueous buffers, and combinations or two or more thereof; and
   (B2) 0.1 to 50% by weight of one or more solid pharmaceutically acceptable carriers, preferably one or more polysaccharides or derivatives thereof or pharmaceutically acceptable salts thereof, in particular polysaccharides or derivatives selected from the group consisting of cellulose derivative (e.g., carboxymethyl cellulose (CMC), carboxyethyl cellulose (CEC)), cellulose, and mixtures of two or more thereof;
(C) 0 to 3% by weight, referred to the injectable composition, of one or more local anesthetics as component C; and
(D) 0 to 50% by weight, referred to the injectable composition, of one or more pharmaceutically acceptable additives other than components A, B, C and D as component D.

Optionally, the injectable composition of the present invention may be packaged. For instance, it may be packaged in syringes (for single use), vials, etc. A user manual may optionally be added to such package. Thus, the present invention also refers to a kit comprising the injectable composition and a user manual for cosmetic and/or therapeutic uses of the present invention.

As indicated above, the calcium hydroxyapatite particles and/or the injectable composition of the present invention may optionally be used for cosmetic (non-therapeutic) and therapeutic purposes. As indicated above, the calcium hydroxyapatite particles and/or the injectable composition of the present invention is particularly usable as a soft tissue filler, in particular a dermal filler. Accordingly, the present invention also relates to the use of the calcium hydroxyapatite particles and/or the injectable composition of the present invention for improving appearance of the skin and/or contour of a part of interest of the face or body of a subject. In particular, the present invention also relates to the use of the calcium hydroxyapatite particles and/or the injectable composition of the present invention as a soft tissue filler, in particular a dermal filler.

The calcium hydroxyapatite particles and/or the injectable composition of the present invention of the present invention may be used to obtain an increased and prolonged collagen production (neocollagenesis). These results indicate that such composition is particularly well suitable for increasing expression of collagen. The calcium hydroxyapatite particles and/or the injectable composition may bear particularly good biostimulation. This may lead to an increased skin quality, which may, for instance, include improvement of wrinkles, skin roughness, skin tightness and/or of signs of aging and facial contouring. It is, thus, a particularly suitable dermal and/or soft tissue filler.

Accordingly, the present invention also relates to the use of the calcium hydroxyapatite particles or the injectable composition of the present invention as a filler, in particular dermal and/or soft tissue filler. The present invention also relates to the use of the calcium hydroxyapatite particles or injectable composition for improving appearance of the skin and/or contour of a part of interest of the face or body of a subject.

A further aspect of the present invention relates to a cosmetic method for improving appearance of the skin and/or contour of a part of interest of the face or body of a subject, said method including the following steps:
(i) providing an injectable composition of the present invention; and
(ii) injecting said injectable composition into the skin of the part of interest of the face or body of a subject.

It will be understood that the definitions and preferred embodiments as laid out in the context of the calcium hydroxyapatite particles and the injectable composition of the present invention mutatis mutandis apply to the cosmetic method.

The present invention also relates to an injectable composition of the present invention for use in a method for improving appearance of the skin and/or contour of a part of interest of the face or body of a subject, said method including the following steps:
(i) providing the injectable composition; and
(ii) injecting said injectable composition into the skin of the part of interest of the face or body.

The term “subject” (also: “individual” or “patient”) may be understood in the broadest to sense as a human or animal, typically a mammal, preferably a human or a domestic mammal, who/which can be subjected to the a cosmetic method or treatment method with the injectable composition of the present invention. As used herein, the term “mammal” may be understood in the broadest sense as any mammalian animal. Preferably, the mammal is a human or a domestic animal such as an animal selected from the group consisting of mouse, rat, cow, pig, dog, cat, horse. Particularly preferably, a subject as used herein is a human. A human or animal administered with the injectable composition of the present invention can also be designated as a patient, independent on his/her health state an irrespective whether clinical symptoms occur or do not occur.

Injecting into the skin of the part of interest of the face or body may be injection in any part of the skin. In one embodiment of the present invention, the composition of the present invention is administered to (in particular injected into) soft tissue. In one embodiment of the present invention, the composition of the present invention is administered to (in particular injected into) the dermis area, such as below the epidermis or above the hypodermis and as such may be injected subcutaneously/subdermally, hypodermically or intradermally, or some combinations. In one embodiment of the present invention, the composition of the present invention is administered (in particular injected) subcutaneously, subdermally, and/or intradermally. In a preferred embodiment, injecting into the skin of the part of interest of the face or body is injecting subcutaneously or intradermally. Injection may be performed by any means such as, e.g., by a syringe.

The purpose of improving appearance of the skin and/or contour of a part of interest of the face or body of a subject may be understood in eth broadest sense.

In a preferred embodiment, the cosmetic or therapeutic method is further characterized in that it is a method for a purpose selected from the group consisting of filling of wrinkles, improving facial lines, breast reconstruction or augmentation, rejuvenation of the skin, buttocks augmentation, remodeling of cheekbones, soft tissue augmentation, filling facial wrinkles, improving glabellar lines, improving nasolabial folds, improving marionette lines, improving buccal commissures, oral commissures, improving peri-lip wrinkles, improving crow's feet, improving subdermal support of the brows, malar and buccal fat pads, improving tear troughs, nose, augmentation of lips, augmentation of cheeks, augmentation of per-oral region, augmentation of scars such as acne scars, augmentation of infraorbital region, resolving facial asymmetries, improving jawlines, augmentation of chin, and combinations of two or more thereof.

In a preferred embodiment, the cosmetic method is a method for filling of wrinkles or improving facial lines, in particular for filling of wrinkles. In a preferred embodiment, the cosmetic or therapeutic method is further characterized in that it is a method for filling of wrinkles of interest of a subject, said method injecting said injectable composition subcutaneously or intradermally into the wrinkles of interest.

In a preferred embodiment, the cosmetic method is a method for filling of wrinkles of interest of a subject, said method including the following steps:
(i) providing an injectable composition of the present invention; and
(ii) injecting said injectable composition subcutaneously or intradermally into the wrinkles of interest.

The above purposes of the cosmetic or therapeutic method may be achieved by any cellular mechanism. In a preferred embodiment, the step (ii) is injecting the injectable composition in connective tissue of the subdermal skin and thereby stimulating the production of collagen, in particular collagen selected from collagen type III, collagen type I, or a combination of collagen type I and III.

As used herein, in the context of a protein such as a collagen type, the term “production” may be understood in the broadest sense as generation of the protein such as one or more collagen types. This may be also understood as protein expression.

As used herein, induction of the production of a collagen type (in particular collagen type I and/or collagen type III) may be understood in the broadest sense as increasing the expression rate by at least 1% by weight, by at least 2% by weight, or by at least 5% by weight, or by at least 10% by weight, or by at least 20% by weight, or by at least 50%, or by at least 100% by weight, in comparison to comparable cells or a comparable tissue not administered with the injectable composition of the present invention.

Collagen (in particular collagen type I and/or collagen type III) is typically generated by fibroblasts. Accordingly, the cells are preferably fibroblasts or the tissue is preferably a tissue containing fibroblasts.

The step of injecting (step (ii)) may be injecting the injectable composition in connective tissue of the subdermal skin. In a preferred embodiment, the step (ii) is injecting the injectable composition in connective tissue of the subdermal skin and thereby stimulating the production of collagen, in particular collagen selected from to collagen type III, collagen type I, or a combination of collagen type I and III.

As indicated above, the calcium hydroxyapatite particles and/or the injectable composition of the present invention may also be used for therapeutic purposes. Accordingly, an aspect of the present invention relates to the calcium hydroxyapatite particles and/or the injectable composition of the present invention for use a medicament.

A further aspect of the present invention relates to the calcium hydroxyapatite particles or the injectable composition of the present invention for use in a method of treating a pathologic condition associated with pathologic deterioration of connective tissue.

In other words, the present invention also relates to a method of treating a pathologic condition associated with pathologic deterioration of connective tissue in a subject, wherein said subject is administered with a sufficient amount of calcium hydroxyapatite particles and/or an injectable composition of the present invention.

A further aspect of the present invention relates to the calcium hydroxyapatite particles or the injectable composition of the present invention for use in a method of substituting or regenerating bone material (i.e., bones and bone grafts), implementing/fixing a tooth root, or filling a tooth.

In other words, the present invention also relates to a method of substituting or regenerating bone material, implementing/fixing a tooth root, or filling a tooth in a subject, wherein said subject is administered with a sufficient amount of the calcium hydroxyapatite particles or the injectable composition of the present invention.

It will be understood that the definitions and preferred embodiments as laid out in the context of the calcium hydroxyapatite particles, the injectable composition and the cosmetic method above mutatis mutandis apply to the injectable composition for use and to the methods of treating.

In a preferred embodiment, the pathologic condition of connective tissue is selected from the group consisting of urinary incontinence, vesicoureteral reflux, vocal cord augmentation, lipotrophy, in particular in a patient suffering from human immunodeficiency virus (HIV), a pathologic condition associated with age-related or pathologic deterioration of connective tissue (also: a pathologic condition associated with age-related deterioration of connective tissue or a pathologic condition associated with pathologic deterioration of connective tissue), and combinations of two or more thereof.

In a preferred embodiment, substituting or regenerating bone material includes the injection of the calcium hydroxyapatite particles or the injectable composition of the present invention into or adjacent to the bone structure to be treated in the subject. Substituting or regenerating bone material can also be bone grafting.

In a preferred embodiment, implementing/fixing a tooth root in includes the administration (e.g., injection) of the calcium hydroxyapatite particles or the injectable composition of the present invention in the anchoring structure of the tooth.

In a preferred embodiment, filling a tooth root in includes the administration of the calcium hydroxyapatite particles or the injectable composition of the present invention in a cavity to be filled in eth tooth (e.g., a naturally occurring cavity of a drilled or milled cavity).

The Figures, Examples and claims described further illustrate the invention.

### BRIEF DESCRIPTION OF THE FIGURES

FIG. 1 shows the size distribution of calcium hydroxyapatite particles sintered at 970° C., which have a mean particle diameter of approximately 30 μm. The relative size distribution (solid line) and the cumulative size distribution (dashed) are depicted. It is visible that the size distribution is rather narrow. The vast majority of particles has a diameter of from 25 to 45 μm.

FIG. 2 shows the comparison of calcium hydroxyapatite particles sintered at 1170° C. (FIG. 2A, comparative example) and those sintered at 970° C. (FIG. 2B, according to the invention) in a microscopic image of 100-fold magnification. The scale bar depicts 100 μm. It is apparent that both sintering temperatures led to particles of essentially spherical shape and well-defined size distribution.

FIG. 3 shows the comparison of calcium hydroxyapatite particles sintered at 1170° C. (FIG. 3A, comparative example) and those sintered at 970° C. (FIG. 3B, according to the invention) in a microscopic image of 500-fold magnification. It is apparent that both sintering temperatures led to particles of essentially spherical shape and well-defined size distribution. Pores (visible as dark spots) can be noted on the surface of the calcium hydroxyapatite particles sintered at 970° C. (FIG. 3B, according to the invention). In contrast, the surfaces of the calcium hydroxyapatite particles sintered at 1170° C. (FIG. 3A, comparative example) are essentially smooth.

FIG. 4 shows the comparison of calcium hydroxyapatite particles sintered at 1170° C. (FIG. 4A, comparative example) and those sintered at 970° C. (FIG. 4B, according to the invention) in a microscopic image of 5200-fold magnification. The scale bar depicts 10 μm. Pores (visible as dark spots) can be noted on the surface of the calcium hydroxyapatite particle sintered at 970° C. (FIG. 4B, according to the invention). In contrast, the surface of the calcium hydroxyapatite particle sintered at 1170° C. (FIG. 4A, comparative example) is essentially smooth.

FIG. 5 shows the mean collagen type III expression per fibroblast cell after 72 hours incubation with different types in terms of size and sintering temperature (970° C., 1070° C. and 1170° C.) of pure calcium hydroxyapaptite (CaHA) particles. The control (CTRL) represents unstimulated fibroblasts incubated at comparable conditions. The fluorescent signal is depicted in arbitrary units (AU).

FIG. 6 shows the collagen type III expression of fibroblasts after incubation with 2 mg/ml of comparable calcium hydroxyapaptite (CaHA) particles of a mean particle diameter of 25 to 45 μm prepared by sintering at different temperatures (1170° C. or 970° C.) after 72 hours of incubation (FIG. 6A) and after 7 days of incubation (FIG. 6B). The control (CTRL) represents unstimulated fibroblasts incubated at comparable conditions. The fluorescent signal is depicted in arbitrary units (AU).

FIG. 7 shows the collagen type I expression of fibroblasts after incubation with 2 mg/ml of comparable calcium hydroxyapaptite (CaHA) particles of a mean particle diameter of 25 to 45 μm by sintering at different temperatures (1170° C. or 970° C.) after 72 hours of incubation (FIG. 7A) and after 7 days of incubation (FIG. 7B). The control (CTRL) represents unstimulated fibroblasts incubated at comparable conditions. The fluorescent signal is depicted in arbitrary units (AU).

FIG. 8 shows an untreated fibroblast cell culture. The grey dots show cells with low collagen type III expression. The black dots indicate cells with high collagen type III expression. It is visible that only two cells of several dozen bear high collagen type III expression.

FIG. 9 shows a fibroblast cell culture treated with calcium hydroxyapaptiude particles having a mean diameter of 25 to 45 μm sintered at 970° C. according to the present invention. The grey dots show cells with low collagen type III expression. The black dots indicate cells with high collagen type III expression. It is visible that a high number of cells bear high collagen type III expression.

FIG. 10 shows the impact on collagen type III expression in a fibroblast cell culture. The cells were treated with particles sintered at different temperatures compared with a control (ctrl) sample. The percentage of cells showing high collagen type III expression (COLIII high cells) is shows (A). Further, the expression of collagen type III (COLIII) per cell showing high collagen type III expression (COLIII high cells) is depicted in comparison to a control sample of untreated cells (Ctrl)set o 100% (B).

### EXAMPLE 1

Preparation and Analysis of Calcium Hydroxyapatite (CaHA) Particles

Preparation of a Slurry of Calcium Hydroxyapatite

a.) Preparation of a Calcium Hydroxyapatite (CaHA) Slurry:

Calcium hydroxyapatite (CaHA) was precipitated via an aqueous slurry as described by Nieh et al. (Nieh, Choi and Jankowski, “Synthesis and characterization of porous hydroxyapatite and hydroxyapatite coatings”, Conference: 2001 Minerals, Metals& Materials Society Annual Meeting & Exhibition, New Orleans, LA (US), Feb. 11-15, 2001). Thus, initially, a crystalline CaHA powder was prepared and precipitated by mixing calcium and phosphorous (e.g., Ca(OH)₂ and H₃PO₄) in a basic aqueous solution having a pH of approximately pH 11 (e.g., by NH₄OH) by mixing. CaHA crystals precipitate at room temperature. The precipitated CaHA slurry was purified by removal of excess reactants and byproducts using de-ionized water as described by Nieh et al. The purified CaHA slurry was concentrated via a decanting process and the CaHA crystals in the slurry were further reduced in size using a process such as a ball mill.

b.) Alternative Preparation of a Slurry Based on Commercial Calcium Hydroxyapatite:

In the present invention, calcium hydroxyapatite powder of a submicron grain size is used. Such calcium hydroxyapatite powder of a submicron grain size is commercially available such as, e.g, from Millipore Sigma and Merck KGaA (Darmstadt, Germany). A slurry of the calcium hydroxyapatite powder is prepared by admixing the powder with water. The content of calcium hydroxyapatite in the slurry is set to 20 to 40% by weight.

c.) Alternative Preparation of a Slurry Based on Generated Calcium Hydroxyapatite Nanocrystals:

3 parts by weight (wt.-parts) of calcium nitrate 4-hydrate are dissolved in approximately 44 wt.-parts of water. 1 wt.-part of diammonium hydrogen phosphate is dissolved in 31 wt.-parts of water. The obtained aqueous solution of diammonium hydrogen phosphate is added slowly to the aqueous solution of calcium nitrate under vigorous stirring. The pH of the obtained solution is adjusted to pH11 by means of sodium hydroxide. The slurry may be aged for several hours. Optionally, the crystal may be washed by one or more centrifugation/washing steps. Such procedure is described in Eslami et al., (Iranian Journal of Pharmaceutical Sciences, 2008, 4(2):127-134). The content of calcium hydroxyapatite in the slurry is set to 20 to 40% by weight.

Preparation and Sintering of Calcium Hydroxyapatite (CaHA) Particles from the Slurry

The CaHA slurry was formed into microspheres utilizing an atomizer/spray dryer as described by Nieh et al. Thus, the slurry is pressed through a nozzle into a warm space. Air classification or mechanical sieving was utilized to remove CaHA particles that are outside the desired diameter threshold. The remaining CaHA particles are sintered as described by Nieh et al. at a temperature of interest and time to control the crystalline structure/porosity of the particles. The sintered CaHA particles were granulated then washed/dried/sieved to achieve a powder consisting of singular CaHA particles of the desired size range.

The preparation of calcium hydroxyapatite (CaHA) particles may also be performed as described in U.S. Pat. No. 6,537,574 and WO 2001/012247.

Analysis of Calcium Hydroxyapatite Particles

Calcium hydroxyapatite particles having a mean particle diameter of from 25 to 45 μm sintered at 970° C. (according to the invention) were compared to comparative particles sintered at 1170° C. (comparative example). The size and shape distribution was analyzed by microscopic means and quantitatively by measuring light scattering.

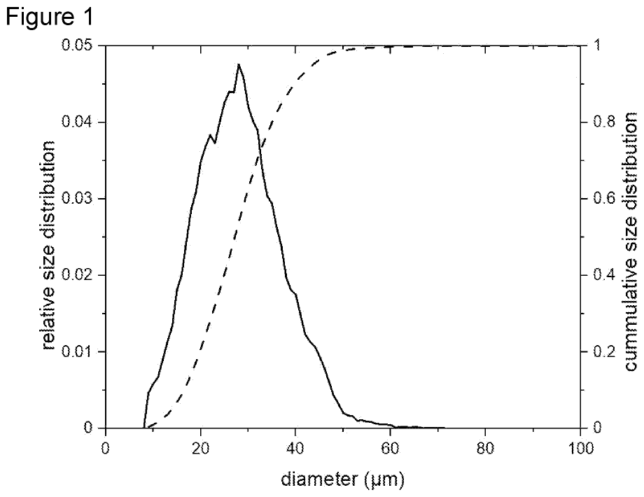

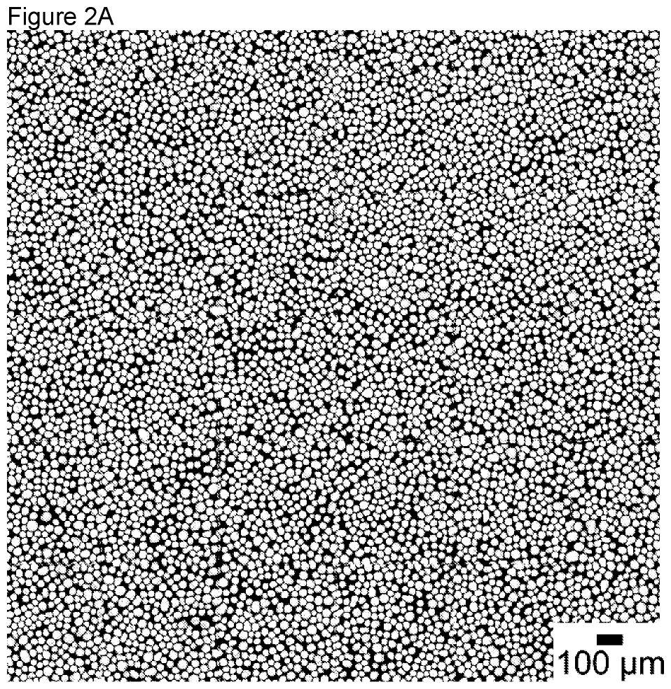

A typical example for a size distribution measurement is depicted in FIG. 1. Herein, it is visible that the size distribution is rather narrow. The vast majority of particles has a diameter of from 25 to 45 μm.

Scanning electron microscopy (SEM) images which were taken from three fractions per sample were used to determine the particle- and volume-weighted size distributions. Therefore, up to 400 SEM images per fraction were taken at a magnification of ×500 and further processed by the automated image analyzing software ImageJ (version 1.51j8). Image processing were provided for every fraction image after defined parameters like Feret diameter and aspect ratio (D-ratio). The averaged results finally represent particle- and volume-weighted size distributions based on at least more than 50,000 identified particles.

Results of a quantitative measurement are depicted in Table 1 below. No particles having a size of >125 μm were found.

TABLE 1
Quantitative comparison of calcium hydroxyapatite (CaHA)
particles having a mean particle diameter of 25 to 45
μm prepared at different sintering temperatures.

| | Sintering temperature of the CaHA particles | | | |
|---|---|---|---|---|
| | 970° C. | | 1170° C. | |
| Number of measures | 66,820 | | 228,776 | |
| particles | | | | |
| Mean area of the outer | 538.9 | (±342.7) | 668.3 | (±271.4) |
| surface of the CaHA | | | | |
| particles in μm² | | | | |
| (±standard deviation) | | | | |
| Feret diameter in μm) | 27.6 | (±9.0) | 30.9 | (±6.6) |
| (±standard deviation) | | | | |
| Perimeter in μm | 93.5 | (±29.2) | 99.0 | (±21.3) |
| (±standard deviation) | | | | |
| D-ratio | 0.9 | (±0.1) | 0.9 | (±0.1) |

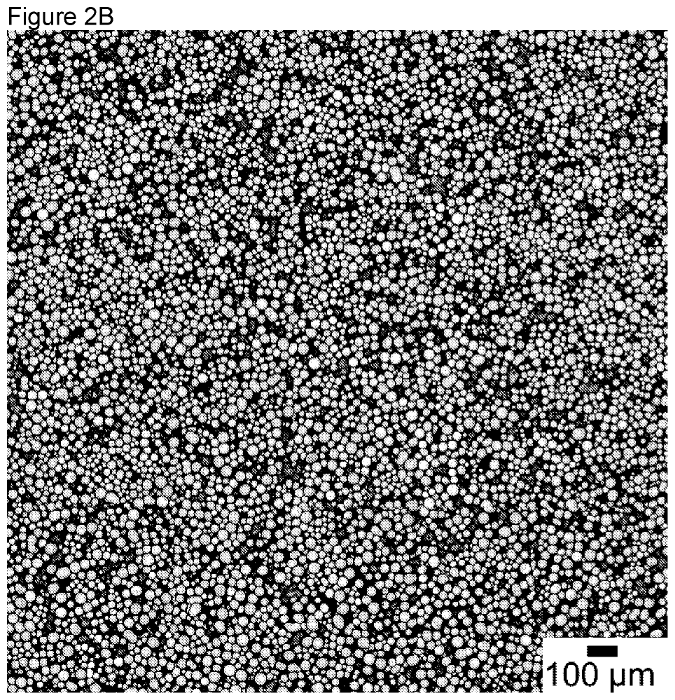

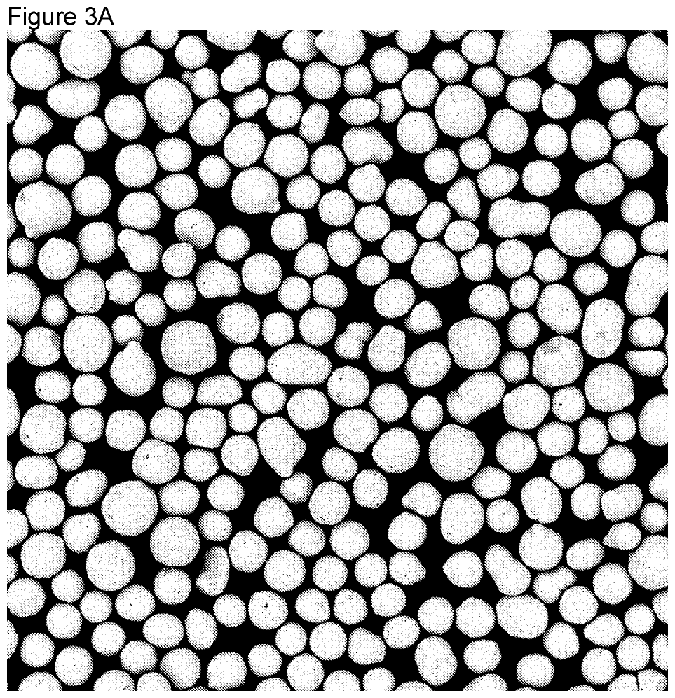

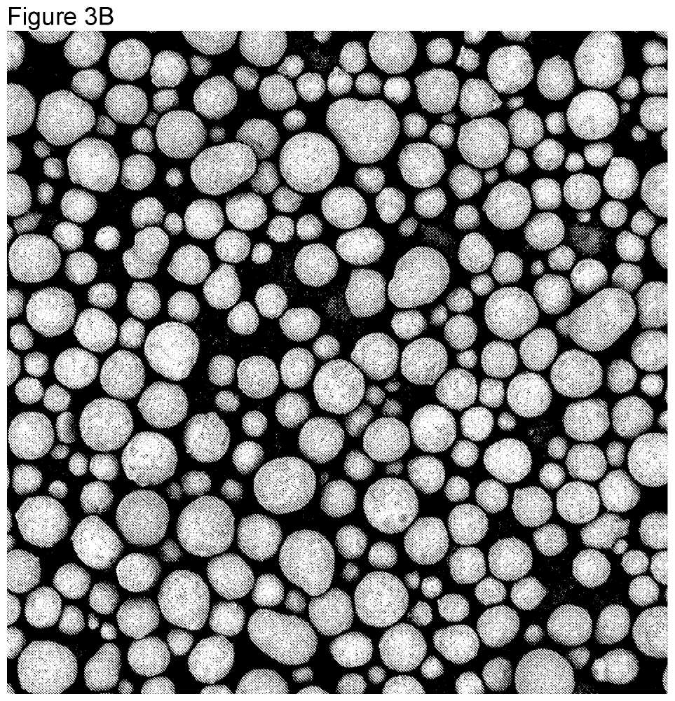

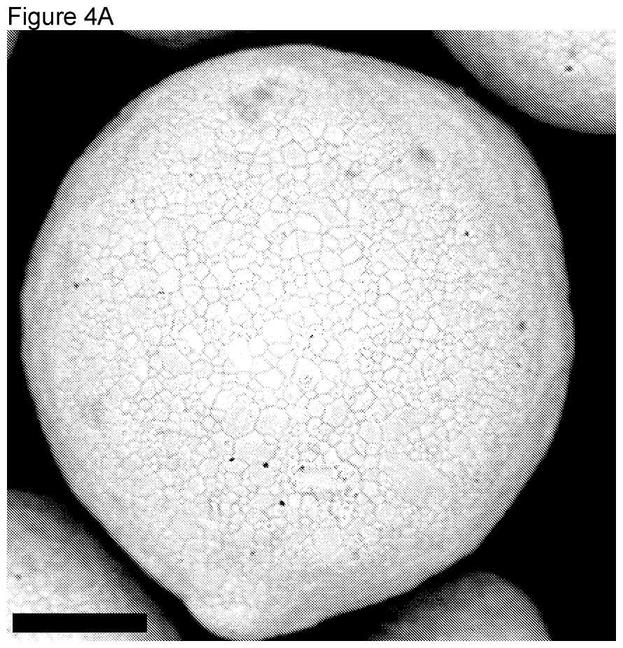

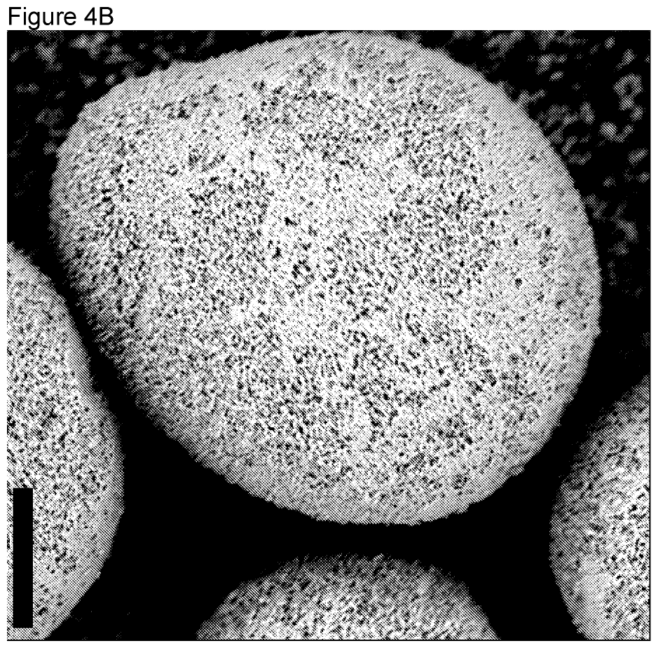

The results are depicted in FIGS. 2 to 4. It is visible that both sintering temperatures led to particles of essentially spherical shape and well-defined size distribution. However, it was surprisingly found that pores (visible as dark spots) can be noted on the surface of the calcium hydroxyapatite particles sintered at 970° C. (according to the invention), while the surfaces of the calcium hydroxyapatite particles sintered at 1170° C. (comparative example) are essentially smooth.

### EXAMPLE 2

Effect of Calcium Hydroxyapatite (CaHA) Particles on Overall Collagen Type I and III Expression of a Cell Culture

Materials and Methods

Materials

Human primary fibroblasts/adult/single donor/breast, PromoCell, #412Z020-P3);
Fibroblast growth medium:cell culture medium including 1 mM vitamin C and 1% by weight of PenStrep (penicillin-streptomycin);
Anti-collagen type III antibody: polyclonal antibody, rabbit, used as primary antibody (Invitrogen, PA5-34787);
Anti-rabbit antibody: AlexaFluor488-labeled secondary antibody, goat, detecting the primary rabbit antibody (Invitrogen, A11034);
Dako antibody solution (Agilent, US);
DAPI: 4′,6-diamidino-2-phenylindole (SIGMA, D9542); and
CellMask: deep red plasma membrane strain (Invitrogen, C10046).

Cell Culture and Sample Preparation

Fibroblasts were seeded at a density of 5000 cells per well. The cells were cultivated for 24 hours at standard conditions at 37° C. in fibroblast growth medium. After 24 hours, 200 μl of the hyaluronic acid-containing samples were added. The samples contained different amounts of hyaluronic acid. Some samples further contained 2 mg/ml calcium hydroxyapatite particles (CaHA).

Cell Culture and Sample Preparation

Human primary fibroblasts (adult, single donor) are used. The cells were cultivated for 24 hours at standard conditions at 37° C. in fibroblast growth medium. After 24 hours of incubation, each 200 μl of a solution containing 2 mg/ml of calcium hydroxyapatite (CaHA) particles sintered at different temperatures (970° C., 1070° C. and 1170° C.) were added. The particles partly also differed in size, starting with <25 μm, 25-45 μm, 45-75 μm, 75-125 μm, and >125 μm. After 72 hours or after 7 days, respectively, the medium was removed from the cells and the cells were fixed with cold methanol (−20° C.) for 10 minutes. Then, the fixed cells were washed three times with phosphate buffered saline (PBS) and stored at 4° C.

Collagen Staining and Quantification:

Two specific antibodies for collagen type III and collagen type I were used. Fixed cells were incubated, and fluorescent signals were analyzed using an Imager for quantification. For collagen type III quantification, the mean expression of cells in the well plate was analyzed, since the signal at the same planar level as the cells. In contrast to that, collagen type I expression was evaluated as mean fluorescent of the whole well since the collagen type I network is forming in a planar level slightly above the cells.

Additionally, single cell analysis was performed for collagen type III expression. Here, every single cell was analyzed individually, and the amount of collagen type III high expressing cells was quantified, and the collagen type III expression of those collagen type III high cells was evaluated.

The supernatant of the fixed cells was removed. Then, the cells were treated with 100 μl/well of a blocking buffer (5% by weight of albumin in PBS) for 2 hours at room temperature (RT). The blocking buffer was removed. Then, 70 μl/well of a solution of the respective anti-collagen antibody (e.g., a solution of 6.7 μg/ml of the primary anti-collagen type III antibody (Anti-Collagen III antibody, polyclonal, host rabbit; L Thermo Fisher Scientific; PA5-34787) in Dako antibody solution (1:100), respectively the anti-collagen type I antibody (Anti-Collagen I antibody [COL-1], monoclonal, host mouse; Abcam; ab90395) in Dako antibody solution (1:100) is used) were added and incubated overnight in the dark at 4° C. on a horizontal mixer.

On the next day, the treated fixed cells were washed three times with PBS. Subsequently, 70 μl/well of a solution of the respective labeled secondary antibody (e.g., containing 10 μg/ml of the secondary AlexaFluo488-labeled anti-rabbit antibody in Dako antibody solution (1:200) and for co-staining AlexaFluo546-labeled anti-mouse antibody in Dako antibody solution (1:200) is added) were added and incubated for 1 hour at RT in the dark. The treated fixed cells were washed three times with PBS. Subsequently, 70 μl/well of CellMask deep red plasma membrane strain were added in a dilution of 1:1000 in PBS (5 μg/ml). The fixed cells were incubated for 30 minutes at RT in the dark. Subsequently, 70 μl/well of a solution of 1 μg/ml DAPI solution in PBS (1 μg/ml, 1:2000 dilution of an aliquot of 2 mg/ml) were added. The fixed cells were incubated for 10 minutes at RT in the dark. The treated fixed cells were washed three times with PBS.

The fluorescence signals of the respective secondary antibody were determined at an Imager for quantification of the signals. Furthermore, microscopic images were prepared. The results are depicted below.

Results

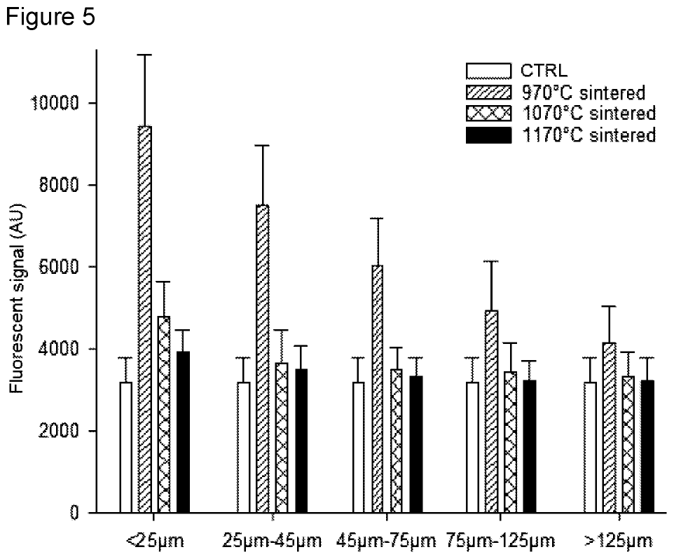

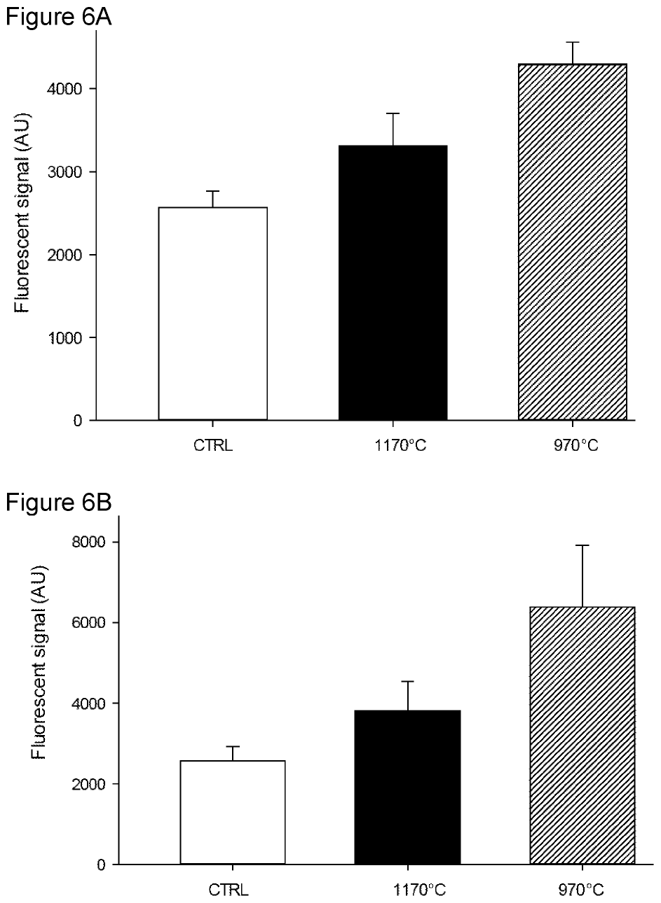

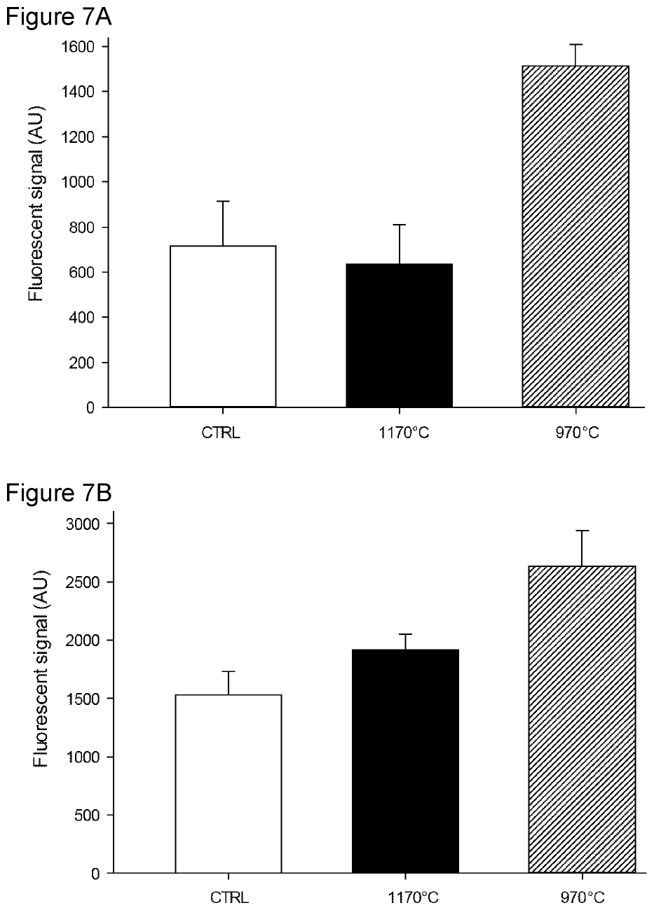

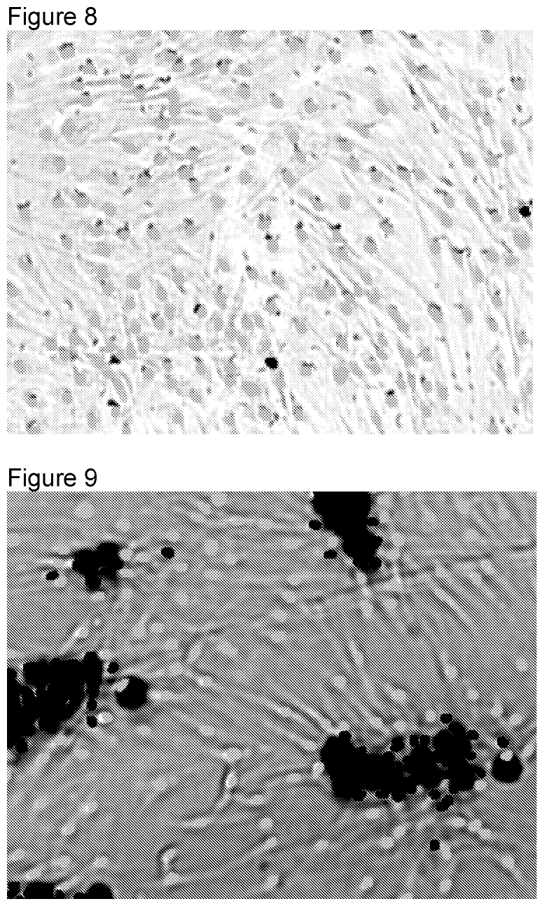

The results are depicted in FIGS. 5 to 7. It was surprisingly found that calcium hydroxyapatite particles sintered at lower temperature (here: 970° C.) showed higher effect on the neocollagenesis, i.e., increase the formation of collagen type I as well as collagen type III. This effect was found in all samples. This effect was particularly significant for collagen type III expression induced by smaller sized particles of a mean particle diameter of not more than 75 μm, in particular not more than 45 μm. Moreover, it was surprisingly found that the generation of collagen type I induced by calcium hydroxyapatite particles sintered at lower temperature (here: 970° C.) was significantly increased after short incubation if 72 hours in comparison to comparative calcium hydroxyapatite particles sintered at 1170° C.

In summary, it was found that the calcium hydroxyapatite particles of the present invention increases neocollagenesis in an efficacy which is superior over calcium hydroxyapatite of the prior art. The particularly efficient increase in collagen production is true for collagen type I and collagen type III, both representing the dominant collagens in the skin and also being the main driver of skin quality improvement. This provides evidence that injectable compositions comprising the calcium hydroxyapatite particles of the present invention can be used as particularly efficient fillers. This may be particularly beneficial for improving appearance of the skin and/or contour of a part of interest of the face or body of a subject.

### EXAMPLE 3

Effect of Calcium Hydroxyapatite (CaHA) Particles on Collagen Type I and III Expression of Cells in a Single Cell Analysis

Methods

A 2D fibroblast cell culture was cultured in a 96-well cell culture plate as described above. Cells were either incubated further without treatment and serve as a control or were treated with a sample of calcium hydroxyapatite particles prepared at a certain sintering temperature and incubated further.

The collagen type I or III was stained as described above. Then, microscopic images are prepared and cells showing high collagen type I or III expression and cells showing low collagen type I or III expression were identified.

Results

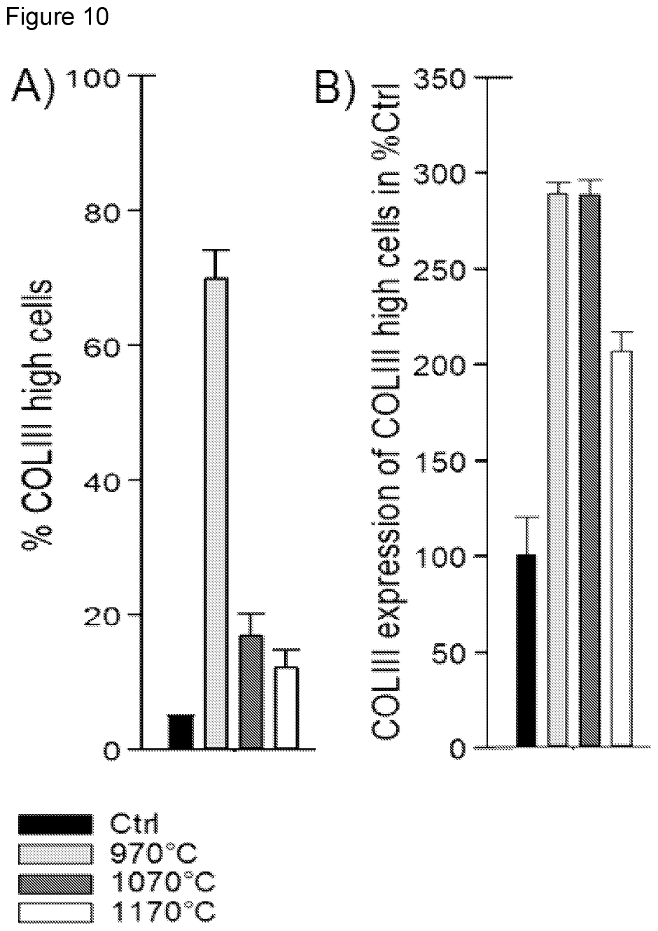

It was surprisingly found that fibroblastic cells treated with calcium hydroxyapatite particles prepared by sintering at 970° C. show a very high number of cells in comparison to the untreated cells and cells treated with comparable calcium hydroxyapatite particles prepared by sintering at higher temperatures (1070° C. and 1170° C.) are expressing high amounts of collagen type III (cf. FIG. 10A). Furthermore, also the collagen type III expression per cell is increased (cf. FIG. 10B).

Therefore, the calcium hydroxyapatite particles of the present invention surprisingly activate a higher number of cells for collagen expression as well as show a higher collagen expression per cell.

### GENERAL EXPERIMENTAL FINDINGS

It was surprisingly found that calcium hydroxyapatite particles of the present invention have a higher porosity compared to particles sintered at higher temperatures. In view of all results, it was surprisingly found that calcium hydroxyapatite particles of the present invention are particularly efficient for stimulating and enhancing the generation of collagen. This was found on a cellular level as well as in the overall cell culture. Collagen formation Collagen generation is associated with a beneficial applicability as a dermal and soft tissue filler (cf. van Loghem et al., The Journal of Clinical Aesthetic Dermatology, 2015, 8(1):38-49; Coleman et al., Dermatologic Surgery, 2008, 34:S53-S55; Berlin et al., Dermatologic Surgery, 2008, 34:S64-S67). The calcium hydroxyapatite particles of the present invention are mainly or completely composed of non-toxic and well-approved calcium hydroxyapatite. Accordingly, it is evident that the calcium hydroxyapatite particles of the present invention are usable as particularly efficient dermal and soft-tissue fillers.

1-15. (canceled)

16. Calcium hydroxyapatite particles, wherein the calcium hydroxyapatite particles are sintered at a temperature in the range of from 910 to 1030° C. and are not subjected to a temperature of more than 1030° C.

17. The calcium hydroxyapatite particles of claim 16, wherein the calcium hydroxyapatite particles are spherical or ellipsoid.

18. The calcium hydroxyapatite particles of claim 16, wherein the calcium hydroxyapatite particles have porous surfaces.

19. The calcium hydroxyapatite particles of claim 16, wherein the surfaces of the calcium hydroxyapatite particles have pores of an average diameter between 10 and 500 nm at the surface as determined by Hg-porosimetry.

20. The calcium hydroxyapatite particles of claim 16, wherein the calcium hydroxyapatite particles have a mean particle diameter from 1 to 500 μm, or from 5 to 500 μm, or from 1 to 150 μm, or from 2 to 100 μm, or from 5 to 80 μm, or from 10 to 60 μm, or from 15 to 50 μm, or from 20 to 45 μm, or from 25 to 45 μm, as determined by light scattering.

21. The calcium hydroxyapatite particles of claim 16, wherein the calcium hydroxyapatite particles are sintered at a temperature in the range from 910 to 995° C., or from 920 to 995° C., or from 930 to 990° C., or from 940 to 985° C., or from 950 to 980° C., or from 960 to 975° C., and wherein the calcium hydroxyapatite particles are not subjected to temperatures above the sintering temperature.

22. The calcium hydroxyapatite particles of claim 16, wherein the calcium hydroxyapatite particles are sintered for 1 to 24 hours, 2 to 12 hours, or 3 to 16 hours.

23. An injectable composition comprising:
(A) one or more types of calcium hydroxyapatite particles of claim 16 as component A;
(B) one or more pharmaceutically acceptable carriers as component B;
(C) optionally one or more local anesthetics as component C; and
(D) optionally one or more pharmaceutically acceptable additives other than components A, B, and C as component D.

24. The injectable composition of claim 23, wherein the one or more pharmaceutically acceptable carriers are selected from the group consisting of one or more polysaccharide derivatives or pharmaceutically acceptable salts thereof, one or more polysaccharides or pharmaceutically acceptable salts thereof, glycerol, water, one or more aqueous buffers, and combinations of two or more thereof.

25. The injectable composition of claim 23, wherein the one or more pharmaceutically acceptable carriers are selected from the group consisting of carboxymethyl cellulose or pharmaceutically acceptable salts thereof, glycerol, water, and combinations of two or more thereof.

26. The injectable composition of claim 23, wherein the injectable composition consists of:
(A) 1 to 80% by weight, based on the total dry matter in the injectable composition, of one or more types of the calcium hydroxyapatite particles as component A;
(B) 1 to 80% by weight, based on the total weight of the injectable composition, of one or more pharmaceutically acceptable carriers as component B comprising at least one pasty, viscous, or liquid carrier;
(C) 0 to 10% by weight, based on the total weight of the injectable composition, of one or more local anesthetics as component C; and
(D) 0 to 50% by weight, based on the total weight of the injectable composition, of one or more pharmaceutically acceptable additives other than components A, B, and C as component D.

27. A cosmetic method for improving the appearance of the skin and/or the contour of a part of interest of the face or body of a subject, the method comprising the following steps:
(i) providing the injectable composition of claim 23; and
(ii) injecting the injectable composition into the skin of the part of interest of the face or body of the subject.

28. The cosmetic method of claim 27, wherein the cosmetic method for improving the appearance of the skin and/or the contour of the part of interest of the face or body of the subject is selected from the group consisting of filling of wrinkles, improving facial lines, breast reconstruction or augmentation, rejuvenation of the skin, buttocks augmentation, remodeling of cheekbones, soft tissue augmentation, filling facial wrinkles, improving glabellar lines, improving nasolabial folds, improving marionette lines, improving buccal commissures, oral commissures, improving peri-lip wrinkles, improving crow's feet, improving subdermal support of the brows, malar and buccal fat pads, improving tear troughs, nose, augmentation of lips, augmentation of cheeks, augmentation of peroral region, augmentation of scars, augmentation of infraorbital region, resolving facial asymmetries, improving jawlines, augmentation of chin, and combinations of two or more thereof.

29. The cosmetic method of claim 27, wherein the step (ii) is injecting the injectable composition in connective tissue of the subdermal skin and thereby stimulating the production of collagen.

30. A method of treating a pathologic condition associated with pathologic deterioration of connective tissue comprising administering the calcium hydroxyapatite particles of claim 16.

31. The calcium hydroxyapatite particles of claim 16, wherein the calcium hydroxyapatite particles are spherical.

32. The calcium hydroxyapatite particles of claim 16, wherein the calcium hydroxyapatite particles are spherical having a D-ratio above 0.7.

33. The cosmetic method of claim 27, wherein the method is a method for filling of wrinkles of interest of the subject, including injecting the injectable composition subcutaneously or intradermally into the wrinkles of interest.

34. A method of treating a pathologic condition associated with pathologic deterioration of connective tissue comprising injecting the injectable composition of claim 23.

35. A method of treating a pathologic condition associated with pathologic deterioration of connective tissue in a patient,
wherein the patient is administered with a sufficient amount of the calcium hydroxyapatite particles of claim 16.
